(19) United States
(12) Patent Application Publication
(10) Pub. No.: US 2008/0146912 A1
(43) Pub. Date: Jun. 19, 2008

(54) INTER-COMMUNICATOR PROCESS FOR SIMULTANEOUS MRI THERMOGRAPHY AND RADIO FREQUENCY ABLATION

(75) Inventors: Howard M. Richard, Columbia, MD (US); Rao P. Gullapalli, Ellicott City, MD (US); Bao Zhang, Cockeysville, MD (US)

Correspondence Address: MERCHANT & GOULD PC, P.O. BOX 2903, MINNEAPOLIS, MN, 55402-0903, US

(73) Assignee: University of Maryland, Baltimore

(21) Appl. No.: 11/957,777

(22) Filed: Dec. 17, 2007

Related U.S. Application Data
(60) Provisional application No. 60/875,503, filed on Dec. 18, 2006.

(51) Int. Cl. A61B 5/055; A61B 18/18

(52) U.S. Cl. 600/411; 606/41

(57) ABSTRACT

The novel method of monitoring radio frequency ablation of cancer tissues by temperature mapping using magnetic resonance thermography, is described. The invention further provides a method of rapid cycling between radio frequency ablation signaling and magnetic resonance image collection that minimizes interference and allows accurate image gathering and effective tissue ablation. Furthermore, the invention provides a method of reducing destruction of healthy surrounding tissue while destroying tumor tissue by radio frequency ablation.

### BACKGROUND OF THE INVENTION

1. Field of the Invention

The present invention relates to the field of medicine, and in particular to the treatment of cancers, including but not limited to breast and prostate cancer, by monitoring of radio frequency ablation using magnetic resonance imaging thermography.

2. Description of the Background Art

The American Cancer Society estimates that 212,920 women will be diagnosed with and 40,970 women will die of breast cancer in the United States in 2006. One in 8 women born today are likely to be diagnosed with breast cancer during their lifetimes. Although these statistics are discouraging, positive trends are evident as a result of innovations in diagnosis and treatment over the past decade. More than 2.5 million women in the U.S. have a history of breast cancer, and a substantial percentage of these women have undergone treatment and are currently disease free. The overall 5-year relative survival rate for breast cancer from 1996 to 2002 was 88.5%, up substantially from only 10 years before. Diagnostic methods, including self-examination, regular mammographic screening, and sophisticated follow-up imaging and biopsy techniques, are among the reasons that 61% of breast cancer cases in this country are diagnosed while the cancer is still confined to the primary (localized) site.

The ability to identify breast cancer at earlier stages and the increasing diagnosis of early-stage breast cancer in younger women have led to a renewed emphasis on less invasive procedures that maximize breast conservation while providing effective treatment strategies and optimal outcomes (1,2). Emerging techniques for minimally invasive (and sometimes noninvasive) in situ treatments of breast cancer include cryoablation, radiofrequency ablation (RFA), microwave thermotherapy, interstitial laser ablation, and focused ultrasound ablation (3-6). One promising technique, ultrasound-guided radiofrequency ablation (RFA), is limited by imaging compromises from microbubbles and by an inability to accurately measure induced hyperthermia.

The majority of investigational studies of RFA in breast cancer have been conducted using ultrasound guidance for needle placement (24,25). A significant limitation of this approach in any RFA application is that RF heating causes gas microbubbles to form in tissues, resulting in considerable acoustic noise/shadowing that impedes the physician's ability to evaluate treatment effect—a crucial capability in achieving maximal extirpation of tumor. Moreover, ultrasound is limited in its ability to detect and assess the temperature changes in tumor and surrounding tissue that signal tumoricidal action in RFA, with resulting complications that range from incomplete tumor destruction to injury to adjacent structures (ie, overlying skin) (9,26).

Porcine mammary tissue has shown promise as a useful in vivo model for developing new breast cancer therapies and for therapies involving heating of fibrofatty tissues. McGahan et al. (26) studied ultrasound-guided RFA in a swine model, reporting successful breast tissue ablation but also describing limitations, including cutaneous erythema.

Imaging, most commonly ultrasound imaging, is used to guide the delivery of radio frequency delivery devices to the target tissue. Ultrasound imaging suffers from a number of disadvantages including poor ability to define the tumor margins, and inability to monitor tissue temperature in real time. These shortcomings of the ultrasound method prevent confident assessment of treatment efficacy at the time of administration, and necessitate the postponement of prognosis until follow up images of the treated area are taken between four and six weeks post treatment.

There is clearly a need to identify alternative image mapping methods which can overcome some of these disadvantages and limitations as observed with ultrasound imaging. MR-guided thermographic mapping offers one such solution and can assist in the achievement of accurate and quantifiable levels of hyperthermia in target breast tissues. Futhermore, this technique may yield optimal ablation of target tissue with minimal damage to surrounding healthy tissue, as monitored by follow-up imaging and pathology.

### SUMMARY OF THE INVENTION

The present invention relates to the use of magnetic resonance (MR) imaging-guided placement of RFA probes. This invention offers a number of improvements in RFA treatments including, but not limited to, (a) a clearer and more reliable picture of the RFA procedure while it is underway, allowing the physician to make sure that the entire tumor is destroyed, and (b) automatic creation of color temperature maps that precisely indicate which tissue is affected.

The present invention also relates to the use of MRI to monitor actual temperatures achieved in target tissues during the treatment procedure in real time. The present invention offers a novel method that allows rapid cycling between MRI and RFA operation to achieve the goal of effective tissue ablation combined with real time temperature measurement that allows confirmation that tissue ablation has been achieved.

The present invention also relates to a method for treatment of cancer in a subject in need of treatment, thereof, the method comprising the destruction of cancerous tissue by radio frequency ablation and the measurement of tissue temperature using magnetic resonance thermography. The magnetic resonance thermography and radio frequency ablation can be executed separately, simultaneously, or sequentially.

Additional advantages and features of the present invention will be apparent from the following drawings and examples, which illustrate preferred embodiments of the invention.

### BRIEF DESCRIPTION OF THE DRAWINGS

FIG. 1. Representative (left) magnitude and (right) phase images of MR thermography of the excised porcine tissue. The region of interest (ROI) is marked by the red circle in the phase image and is adjacent to the thermocouple tip.

FIG. 2. Temperature measured by independent thermocouple (blue diamonds) and phase shift (pink squares) of the ROI obtained by MRI. The thermal coefficient (−0.0107 ppm/⁰C; r²=0.91) was calculated based on correlation between temperature and phase difference when RF was off. Linear regression of the temperature determined by the thermocouple and the phase was also performed while the RF was on. Although the r² value (0.55) was poor, the calculated thermal coefficient remained almost the same. More effort may be needed to reduce RF noise levels.

FIG. 3. Representative color-coded temperature maps corresponding to points marked by red diamonds in the temperature-time curve in FIG. 5 (≧60° C.=red, 41° C.-59° C.=yellow, and ≦40° C.=green).

### DETAILED DESCRIPTION OF THE INVENTION

Reference will now be made in detail to the presently preferred embodiments of the invention, which, together with the drawings and the following examples including prophetic examples, serve to explain the principles of the invention. These embodiments are described in detail to enable those skilled in the art to practice the invention, and it is to be understood that other embodiments may be utilized without departing from the spirit and scope of the present invention. Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, the preferred methods, devices and materials are now described.

Identification of breast cancer at earlier stages and increasing diagnoses in younger women have led to a renewed emphasis on less invasive procedures that maximize conservation while providing effective treatment. A number of new techniques are used to treat early stage breast cancer with maximum effectiveness and conservation of healthy breast tissue but without full surgical intervention. One promising technique, ultrasound-guided radiofrequency ablation (RFA), is limited by imaging compromises from microbubbles and by an inability to accurately measure induced hyperthermia.

Imaging, most often ultrasound, is used to guide the delivery of RF to highly targeted areas of tissue. RF energy causes the tissues to become heated, destroying tumor and sparing surrounding healthy tissue.

RFA is one of a number of new techniques used to treat early stage cancers including breast and prostate cancers. This procedure allows effective non-surgical intervention with conservation and preservation of healthy tissue. Presently, RFA is not monitored in terms of actual temperatures achieved in target tissues during the treatment procedure. Efficacy of the RFA treatment protocol is currently not established until follow-up imaging is carried out at four (4) to six (6) weeks after the treatment.

Magnetic resonance imaging (MRI) can allow real time monitoring of radio frequency ablation (RFA). However, there is significant interference to the acquisition of the MRI imaging information while RFA is in progress. Therefore, there is a need for an automated process that allows the MRI and RFA procedures to cycle rapidly so that MRI can be used to guide the RFA procedure and monitor its progress.

MRI offers the ability to monitor actual temperatures achieved in target tissues during the treatment procedure in real time. However, interference problems arise if the MRI and RFA machines are operated simultaneously. The present invention offers a novel method that allows rapid cycling between MRI and RFA operation to achieve the goal of effective tissue ablation combined with real time temperature measurement that allows confirmation that tissue ablation has been achieved.

The present invention relates to the use of magnetic resonance (MR) imaging-guided placement of RFA probes. This invention offers a number of improvements in RFA treatments including, but not limited to, (a) a clearer and more reliable picture of the RFA procedure while it is underway, allowing the physician to make sure that the entire tumor is destroyed, and (b) automatic creation of color temperature maps that precisely indicate which tissue is affected.

MR-guided thermographic mapping can assist in the achievement of accurate and quantifiable levels of hyperthermia in target breast tissues. Futhermore, this technique may yield optimal ablation of target tissue with minimal damage to surrounding healthy tissue, as monitored by follow-up imaging and pathology.

Percutaneous RFA has been widely applied with safety and success in treatments of hepatocellular and other liver lesions and in renal tumors (7-9). In these applications, CT, ultrasound, or magnetic resonance (MR) imaging is used to guide the placement of a needle(s) directly into the tumor for delivery of RF energy and achievement of local hyperthermia. Several pilot studies of RFA techniques in breast tumors (both in vitro and in animal and human studies) have shown promise (10-14). In one study, ultrasound-guided RFA performed in patients immediately before surgical resection resulted in coagulative necrosis of 96% of resected tumor with a very low complication rate (15,16). Another study reported similar success and also noted that postablation MR imaging was predictive of histologic findings at delayed resection (17). Promising results have been reported in ultrasound-guided RFA of small tumors (≦2 cm) in patients scheduled for lumpectomy or mastectomy (18,19). Most recently, groups have reported on success in RFA in breast tumors in both animal research and humans, particularly when combined with radiation therapy (10) or with adjuvant chemotherapy (21). Encouraging reports of palliative effects (22) and improvements in quality of life (23) after RFA in breast cancer have also appeared in the literature.

MR imaging, which has been used with RFA in hepatic and other cancers, is not subject to these limitations (27,28). MR guidance has several advantages, including: (a) near real-time visualization with no ionizing radiation burden (29,30); and (b) interference-free MR temperature-mapping techniques that provide the ability to directly visualize temperature changes in 3 dimensions, so that the extent of tumor destruction is apparent and the physician can iteratively modify treatment to ensure maximum effectiveness (31,32). This approach is suitable in breast tissues, which offer access for RFA and imaging with no interference with lungs or major vessels.

### EXAMPLES

### Example 1—MR Thermography Assessment of RFA

A breast phantom was developed for initial proof-of-concept MR thermography studies. The cylindrical phantom with a radius of 6 cm was designed to mimic the human breast in geometric, mechanical, and biochemical aspects as well as in T1/T2 relaxivity (33-37). Small spherical inclusions (radius, 1˜2 cm) and irregular-shape inclusions (˜2 cm) were inserted to mimic fibroglandular tissue and tumors. Tumor inserts also included 10 mmol/L of choline to mimic the metabolite abnormality consistent with tumor. For initial calibration of MR thermography, a homogeneous breast phantom was created.

MR Thermography of RFA

MR guidance was used for targeting lesions in the phantoms. For each study, the phantom was placed into a dedicated 4-channel open breast coil (MedRad, Inc.; Indianola, Pa.), fitted with a Suros Biopsy grid system (Suros Surgical Systems, Inc.; Indianapolis, Ind.). MR imaging was performed on a 3-T MR imaging unit (Siemens Medical Solutions; Malvern, Pa.). Targeting of focal abnormalities was facilitated by targeting software from DynaCad software (Invivo; Orlando, Fla.). A flexible MR-compatible needle (RITA Medical Systems, Inc.; Fremont, Calif.) was used for RFA. The curved probe of the RF needle was ideally suited for use with the Suros Biopsy grid system, and the probe gave off minimal artifact when placed in the phantom.

MR thermography was performed by measuring first the proton resonance frequency shift (proportional to the temperature change [38]), which resulted in phase images depending on the temperature of the tissue (39). Subtraction of reference (a phase image with uniform temperature distribution) from objective phase images enabled the generation of phase difference maps. These phase difference maps were converted to temperature maps based on the thermal coefficient of the proton chemical shift resulting from temperature change.

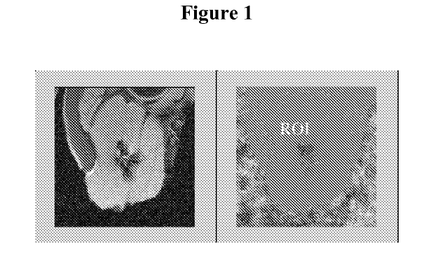

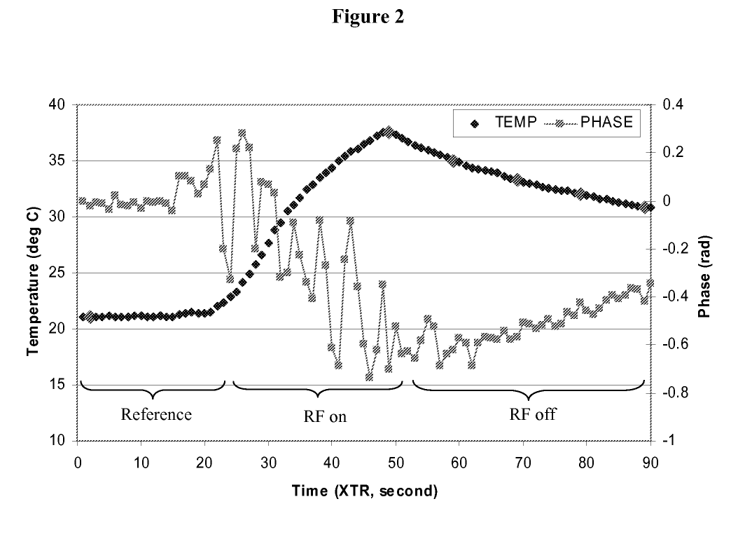

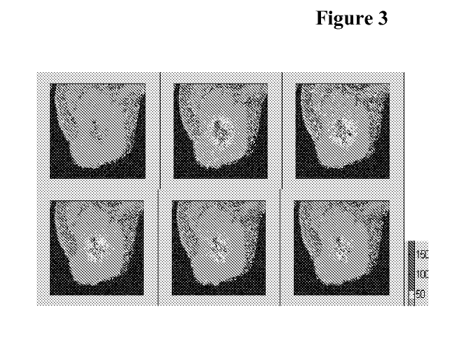

Temperature was also measured independently during RFA with a digital thermometer (accurate to 0.1° C.) placed into the phantom at the location of the RFA. Temperature measured by the independent thermometer was correlated with the phase difference of a region of interest (ROI) very close to the thermocouple tip in the phase images. A thermal coefficient was calculated using linear regression between the thermocouple-determined temperature and the phase. Okuda et al. (39) calculated a coefficient of −0.0110 ppm/° C. on bovine liver on a Signa Horizon Echospeed MR unit. We calculated a coefficient of −0.0116 ppm/° C. (r²=0.96) on the breast phantom on our Siemens 3.0 T MR unit. The minor differences in coefficients between our study and Okuda's may be attributed to differences in MR pulse sequences used. The MR imaging process generated phase difference maps at a temporal resolution of 10.4 s (MR acquisition TA) during the ablation. Phase difference maps were converted to temperature maps based on the coefficient above. Each voxel in the temperature maps was then assigned a color based on temperature. In our proposed study, the MR thermography zone with temperatures ≧60° C. will be considered the region that has been effectively treated by RFA, and the size will be measured in 3D with a spatial resolution of 0.94×0.94×4 mm³ (voxel size). MR thermography was then performed on excised porcine tissue (FIGS. 1-3).

### Example 2 [Prophetic]—R-FA in Swine Breast Tissue

One difficulty in measuring temperatures in phantoms is related to liquification. The phantom liquifies at 40° C. Traditional RFA techniques rely on heating the RF probe to 100° C. and allowing the heat to dissipate into the surrounding tissues so that the entire ablation zone achieves a temperature >60° C. for the tumoricidal effect. The breast phantom is fundamentally limited in this regard. Swine mammary tissue and human breast tissue explants will be studied in vitro. The effects of RFA with regard to the propensity of breast tissue to liquify when heated with RFA can be determined. This phenomenon has been alluded to by Bohm et al. (40), who demonstrated irregular expansion of RF lesions as a result of liquefying fat.

Specimens of swine mammary tissue can be obtained as discards from meat processing (Gwaltney, Inc.; Smithfield, Va.) and preserved on ice in transit. Each specimen can be placed into a dedicated 4-channel open breast coil (MedRad, Inc.; Indianola, Pa.), fitted with a Suros Biopsy grid system (Suros Surgical Systems, Inc.; Indianapolis, Ind.). MR imaging guidance will be used to target the center of each sample and performed on a 3T MR imaging unit (Siemens Medical Solutions; Malvern, Pa.). Targeting of the center of the specimen will be facilitated by targeting software from DynaCad software (Invivo; Orlando, Fla.). An MR-compatible RF needle (RITA Medical Systems, Inc.; Fremont, Calif.) can be placed into the center of each sample, and a 3-cm ablation performed according to the RITA protocol. The curved probe of the RF needle is ideally suited for use with the Suros Biopsy grid system. The electrode will heat to a target temperature of 100° C. and maintain that temperature for 5 min. The MR imaging process will generate temperature maps at 1-min intervals during the ablation.

Outcome variables of size and volume of the predicted ablation zone can be determined by measuring the size and volume of the area on the MR thermography temperature map in which a temperature ≧60° C. is achieved. A second set of outcome variables will be the size and volume of imaging changes as seen on the T2-weighted MR images. For size, an important measure is the short-axis length of the ablation zone, which represents the smallest adequately treated dimension. MR thermography volumes can be calculated by counting the number of voxels with a temperature >60° C. and multiplying by the size of the voxel. In addition, T2-weighted MR image change volumes can be measured by the planimetry volume (PV) technique. Axial images can be used for volume measurement. In the PV technique, areas of change consistent with the RFA can be manually traced with the cursor on a slice-by-slice basis and multiplied by slice thickness.

Breast tissue specimens can be sliced (3-mm thick) and then photographed. Digital photographs can be correlated with MR images, MR temperature maps, and pathology results in a fashion similar to that currently employed for evaluation of prostate specimens. The size of the central zone of white coagulation and the peripheral zone of red coagulation, as well as the short-axis length is measured. The size of the ablation zone can be measured in 3 orthogonal planes and the volume calculated using the equation for a prolate ellipse (W×H×L×0.523).

Statistical Analysis.

The Wilcoxon signed rank (nonparametric) test can be used to compare size and volume measurements. The shortest diameter, x, y, and z orthogonal plane measurements, and the volume of the predicted ablation zone as determined from the MR temperature map will be compared with the size and volume of the ablation zone of coagulation as determined from the size of pathologic coagulation. The Wilcoxon signed rank (nonparametric) test can be used to compare the size of ablation with results from the traditional T2-weighted MR images. It is predicted that the zone of ablation from the temperature maps generated in this process will be equivalent to the actual zone of ablation seen in the breast tissue. It is a working hypothesis that the the MR thermography-predicted zone of ablation can be used to reliably predict the actual zone of ablation.

### Example 3 [Prophetic]—RFA in Human Breast Tissue

Specimens of human breast tissue can be obtained from a tissue bank service. Specimens can be preserved on ice in transit. Ablation, MR thermography, MR imaging, photography, and pathology and results analysis can be performed as described previously for swine tissue.

Statistical Analysis.

Statistical analysis will be the same as described previously for swine tissue results. Such analyses will allow for the evaluation of the system based on large differences in effect and therefore for estimating the effect size.

### Example 4 [Prophetic]—Comparison of Swine and Human Tissue Results

Comparison of the results of RFA in the swine model with those in the human breast tissues can be used for preparation of in vivo experiments with RFA in swine. It is expected that the size of the ablations will be the same in both the swine and human breast tissue. It is expected that the ability of the MR thermography maps can be used to predict the size of the ablation zone in human and swine breast tissue. The Wilcoxon signed rank (nonparametric) test can be used to compare size and volume measurements in the tissues. If a trend is noted in which MR thermography yields different results in RFA assessment in swine and human breast tissue, then it is expected that these difference are representative of differences between RFA in the in vivo swine model and clinical applications in patients.

### Example 5 [Prophetic]—In Vivo R-FA Imaging In Swine

Healthy adult female pigs can be obtained from an animal supplier. All procedures for housing and treatment should be in accord with our IACUC policies, which closely follow the PHS Policy on Humane Care and Use of Laboratory Animals, amended Animal Welfare Act requirements, and other federal statutes and regulations relating to animals. Animals can be pretreated with intravenously administered domosedan in the stall, followed by thiopental (50 mg/kg body weight) at the MR unit. Local anesthesia can also be administered at RFA sites. After RFA, each animal is administered with a halothane washout to re-establish spontaneous respiration.

Four RFAs can be performed per animal. MR imaging guidance can be used to target the center of each sample and is performed on a 3T MR imaging unit (Siemens Medical Solutions). Targeting of the center of each specimen can be facilitated by targeting software from DynaCad (Invivo). Suros Atec-13 biopsy marker clips can be used to mark the MR-compatible ablation sites. The first Suros Atec-13 biopsy marker clip is placed 2-cm deep to the anticipated ablation target. An MR-compatible RF needle (RITA Medical Systems, Inc.) is be placed into the center of each sample, and a 3-cm ablation can be performed according to the RITA protocol. The electrode will heat to a target temperature of 100° C. and maintain target temperature for 5 min. The MR process will generate temperature maps at 1-min intervals during the ablation. A second Suros Atec-13 biopsy clip will be placed 2-cm proximal to the center of the ablation lesion. The biopsy clips then will be 4 cm apart, bracketing the 3-cm ablation lesion.

Outcome variables of the size and volume of predicted ablation zone can be determined by measuring the size and volume of the area on the MR thermography map in which a temperature ≧60° C. is achieved. A second set of outcome variables will be the size and volume of the imaging changes as seen on the T2-weighted MR images. As noted, an important measure is the short-axis length of the ablation zone (smallest adequately treated dimension). MR thermography volumes can be calculated by counting the number of voxels with a temperature >60° C. and multiplying by the size of the voxel. In addition, T2-weighted MR image change volumes will be measured by the PV technique. Axial images can be used for determining volume measurement.

Animals can be euthanized (KCl 15 mg IV, sodium pentobarbital [Narcoren] 10 mL IV) per institutional procedure immediately after the procedure (n=3), at 1 week (n=3), and at 2 weeks (n=3) after ablation to evaluate pathologic features of RFA in the acute, subacute, and chronic phases, respectively. The image plane can then be correlated by the skin entry site and biopsy markers. Breast specimens can be sliced and then photographed. Digital photographs are correlated with MR images, MR temperature maps, and pathologic evaluation. Pathologic features can be examined by hematoxylin-eosin staining (HE). Viability can be evaluated with α-nicotinamide adenine dinucleotide diaphorase (NADD) staining.

The size of the central zone of white coagulation and the peripheral zone of red coagulation can be measured. The short-axis length can be measured, and the size of the ablation zone can also be measured in 3 orthogonal planes and the volume calculated using the equation for a prolate ellipse (W×H×L×0.523). The size of the ablation lesion (ie, the size of the central zone of white coagulation and the peripheral zone of red coagulation) can be measured using HE, NADD in 3 orthogonal planes. These results can then be compared with imaging changes on the T2-weighted MR images and MR thermography. In addition, the animals can be evaluated for any adverse effects, such as skin damage or infection.

The goal is to be able to deliver RFA to the tissue and monitor the temperature of the tissue during the ablation process. It is predicted that temperature monitoring is reliable to detect temperature differences ≦1° C. During this process various means can be identified by which the procedure can be optimized through the use of feedback loops to the scanner and ablation system.

Statistical Analysis.

Statistical analyses are the same as described previously for swine tissue results.

### REFERENCES

1. Singletary S E. Minimally invasive surgery in breast cancer treatment. Biomed Pharmacother. 2001; 55:510-514.
2. Noguchi M. Minimally invasive surgery for small breast cancer. J Surg Oncol. 2003;84:94-101; discussion 102.
3. Sabel M S, Edge S B. In-site ablation of breast cancer. Breast Dis. 2001;12:131-140.
4. Hall-Craggs M A, Vaidya J S. Minimally invasive therapy for the treatment of breast tumours. Eur J Radiol. 2002;42:52-57.
5. Agnese D M, Biurak W E Jr. Ablative approaches to the minimally invasive treatment of breast cancer. Cancer J. 2005; 11:77-82.
6. Huston T L, Simmons R M. Ablative therapies for the treatment of malignant diseases of the breast. Am J Surg. 2005;189:694-701.
7. Brown D B. Concepts, considerations, and concerns on the cutting edge of radiofrequency ablation. J Vasc Interv Radiol. 2005;16:597-613.
8. Gillams A R. The use of radiofrequenct in cancer. Br J Cancer. 2005;92:1825-1829.
9. Livraghi T, Solbiati L, Meloni M F, Gazelle G S, Halpern E F, Goldberg S N. Treatment of focal liver tumors with percutaneous radiofrequency ablation: complications encountered in a multicenter study. Radiology. 2003;226:441-451.
10. Noguchi M. Radiofrequency ablation treatment for breast cancer to meet the next challenge: how to treat primary breast tumor without surgery. Breast Cancer. 2003; 10:1-3.
11. Jeffrey S S, Birdwell R L, Ikeda D M, et al. Radiofrequency ablation of breast cancer: first report of an emerging technology. Arch Surg. 1999;134:1064-1068.
12. Elliott R L, Rice P B, Suits J A, Ostrowe A J, Head J F. Radiofrequency ablation of a stereotactically localized nonpalpable breast carcinoma. Am Surg. 2002;68:1-5.
13. Noguchi M, Earashi M, Fujii H, Yokoyama K, Harada K, Tsuneyama K. Radiofrequency ablation of small breast cancer followed by surgical resection. J Surg Oncol. 2006;93:120-128.
14. Singletary S E, Fornage B D, Sneige N, et al. Radiofrequency ablation of early-stage invasive breast tumors: an overview. Cancer J. 2002;8:177-80.
15. Izzo F, Thomas R, Delrio P, et al. Radiofrequency ablation in patients with primary breast carcinoma: A pilot study in 26b patients. Cancer. 2001; 92:2036-2044.
16. Izzo F, Thomas R, Delrio P, et al. Radiofrequency ablation in patients with primary breast carcinoma: a pilot study in 26 patients. Cancer. 2001;92:2036-2044.
17. Burak W E, Agnese D M, Povoski S P, et al. Radiofrequency ablation of invasive breast carcinoma followed by delayed surgical excision. Cancer. 2003;98:1369-1376.
18. Fornage B D, Sneige N, Ross M I, et al. Small (<or =2-cm) breast cancer treated with US-guided radiofrequency ablation: feasibility study. Radiology. 2004;231:215-224.
19. Singletary SE. Radiofrequency ablation of breast cancer. Am Surg. 2003;69:37-40.
20. Horkan C, Dalal K, Coderre J A, et al. Reduced tumor growth with combined radiofrequency ablation and radiation therapy in a rat breast tumor model. Radiology. 2005;235:81-88.
21. Ahmed M, Goldberg S N. Combination radiofrequency thermal ablation and adjuvant IV liposomal doxorubicin increases tissue coagulation and intratumoural drug accumulation. Int J Hyperthermia.2004;20:781-802.
22. Vansonnenberg E, Shankar S, Parker L, et al. Palliative radiofrequency ablation of a fungating, symptomatic breast lesion. AJR Am J Roentgenol. 2005;184(3 suppl):S126-S128.
23. Roberts J, Morden L, MacMath S, et al. The quality of life of elderly women who underwent radiofrequency ablation to treat breast cancer. Qual Health Res. 2006; 16:762-772.
24. Hayashi A H, Silver S F, van der Westhuizen N G, et al. Treatment of invasive breast carcinoma with ultrasound-guided radiofrequency ablation. Am J Surg. 2003; 185:429-435.
25. Boehm T, Malich A, Reichenbach J R, Fleck M, Kaiser W A. Percutaneous radiofrequency (RF) thermal ablation of rabbit tumors embedded in fat: a model for RF ablation of breast tumors. Invest Radiol. 2001;36:480-486.
26. McGahan J P, Griffey S M, Schneider P D, et al. Radio-frequency electrocautery ablation of mammary tissue in swine. Radiology. 2000;217:471-476.
27. Huppert P E, Trubenbach J, Schick F, Pereira P, Konig C, Claussen C D. MRI-guided percutaneous radiofrequency ablation of hepatic neoplasms: first technical and clinical experiences. [in German]. Rofo. 2000;172:692-700.
28. Gaffke G, Gebauer B, Gnauck M, et al. Potential advantages of the MRJ for the radiofrequency ablation of liver tumors [in German]. Rofo. 2005;177:77-83.
29. Lewin J S, Nour S G, Connell C F, et al. Phase II clinical trial of interactive MR imaging guided interstitial radiofrequency thermal ablation of primary kidney tumors: initial experience. Radiology. 2004;232:835-845.
30. Kacher D F, Jolesz F A. MR imaging-guided breast ablative therapy. Radiol Clin North Am. 2004;42:947-962.
32. Scheffler K. Fast frequency mapping with balanced SSFP: Theory and application to proton-resonance frequency shift thermometry. Magn Reson Med. 2004; 51:1205-1211.
32. Vigen K K, Jarrard J, Rieke V, et al. In vivo porcine liver radiofrequency ablation with simultaneous MR temperature imaging. J Magn Reson Imaging. 2006;23:578-584.
33. Rakow R, Danniel B, Sawyer-Glover A, Glover G. T1 and T2 measurements of breast tissue at 0.5T, 1.5T and 3.0T. International Society for Magnetic Resonance in Medicine, Toronto Canada 10-16 Jul., 2003 (ISMRM), 2003.
34. Eliat P A, et al. Magnetic resonance imaging contrast-enhanced relaxometry of breast tumors: an MRI multicenter investigation concerning 100 patients. Magp Res Imaging. 2004;22:475-481.
35. Lorenzen J, Sinkus R, Lorenzen M, et al. MR elastography of the breast: preliminary clinical results. Rofo. 2002;174:830-834.
36. Zhang B. Measurement of the shear modulus of tissue-like materials using magnetic resonance elastography (MRE). Thesis, 2001.
37. Yoshimura K, et al. Development of a tissue-equivalent MRI phantom using carrageenan Gel. MRM 2003.
38. Weidensteiner C, Quesson B, Caire-Gana G, et al. Real time MR temperature mapping of rabbit liver in vivo during thermal ablation. Magn Reson Med. 2003;50:322-330.
39. Okuda S, Kuroda K, Kainuma O, et al. Accuracy of MR temperature measurement based on chemical shift change for radio frequency ablation using hook-shaped electrodes. Magn Res Med Sci. 2004;3:95-100.
40. Bohm T, Hilger I, Muller W, Reichenbach J R, Fleck M, Kaiser W A. Saline-enhanced radiofrequency ablation of breast tissue: an in vitro feasibility study. Invest Radiol. 2000;35:149-157.

What is claimed is:

1. A method for the treatment of cancer in a subject in need of such treatment comprising the destruction of cancerous tissue by radio frequency ablation and the measurement of tissue temperature using magnetic resonance thermography.

2. The method of claim 1, wherein said cancer is breast cancer.

3. The method of claim 1, wherein said cancer is prostate cancer.

4. The method of claim 1, wherein said radio frequency ablation and said magnetic resonance thermography are executed separately.

5. The method of claim 1, wherein said radio frequency ablation and said magnetic resonance thermography are executed simultaneously.

6. The method of claim 1, wherein said radio frequency ablation and said magnetic resonance thermography are executed sequentially.

7. The method of claim 6, wherein said radio frequency ablation and said magnetic resonance thermography are repeatedly executed sequentially.

8. A method for performing simultaneous magnetic resonance imaging thermography and radio frequency ablation comprising measuring the proton resonance frequency shift to create an objective phase image, subtracting a reference image, characterized by uniform temperature distribution, from the objective phase image, and generating phase difference maps which can be use construct temperature difference maps.

9. The method of claim 9, wherein said temperature maps are used to predict actual zones of ablation in a tissue.

10. The method of claim 9, wherein said tissue is human breast tissue.

11. The method of claim 9, wherein said tissue is human prostate tissue.

12. The method of claim 8, wherein said temperature difference is about 1° C.

13. The method of claim 8, wherein said magnetic resonance thermography provides real time visualization and interference free magnetic resonance temperature mapping.

14. The method of claim 13, wherein said temperature mapping can be visualized in three dimensions.
